(19) United States
(12) Patent Application Publication
(10) Pub. No.: US 2020/0370065 A1
(43) Pub. Date: Nov. 26, 2020

(54) SALT-TOLERANT PLANTS

(71) Applicant: BRENT L. NIELSEN, OREM, UT (US)

(72) Inventor: BRENT L. NIELSEN, OREM, UT (US)

(21) Appl. No.: 16/879,722

(22) Filed: May 20, 2020

Related U.S. Application Data
(60) Provisional application No. 62/850,363, filed on May 20, 2019.

(51) Int. Cl. C12N 15/82; A01C 1/06; A01G 7/06; A01H 17/00

(52) U.S. Cl. CPC C12N 15/8273; A01H 17/00; A01G 7/06; A01C 1/06

(57) ABSTRACT

A salt tolerant plant is created by inoculation of a glycophyte plant with bacteria isolated from a halophyte plant.

### CROSS REFERENCE TO RELATED APPLICATIONS

Priority is claimed from United States Provisional Patent Application 62/850,363, filed May 20, 2019, which is hereby incorporated by reference.

### SUMMARY

Many agricultural areas in the southwestern United States and other parts of the world rely heavily on irrigation. In many of these areas soil salinity has been increasing due to drought combined with poor irrigation practices. Most crop plants are sensitive to salt, which leads to reductions in production (reviewed in Gul et al., 2014). The severity of increasing soil salinity will likely intensify with growing food demand and degradation and loss of prime agricultural land. According to the USDA salinity laboratory website (https://www.ars.usda.gov/pacific-west-area/nverside-ca/us-salinity-laboratory/), about 15% of cultivated land globally is irrigated, but irrigated areas account for up to 40% of the total food harvest. In the U.S., salinity of soil and water affects about 30% of all irrigated land, while about 50% of irrigated land worldwide is affected. Salinity increases in irrigated areas due to soluble salts carried in the irrigation water that remain in the soil after evaporation and transpiration. Unless these salts are leached from the soil, they accumulate to levels that are inhibitory to plant growth and may lead to soils becoming sodic, causing degradation of soil structure to affect water and root penetration along with other problems (Gul et al., 2014). According to USDA estimates, about 10 million hectares are lost globally each year as a result of salinity and/or waterlogging. Salinity and other environmental stresses will require new approaches to maintain an adequate food supply.

There have been attempts to use halophiles isolated from the rhizosphere or as endophytes from halophytes as inocula to stimulate the growth of salt-sensitive crops with varied success as evidenced in a number of publications on plant growth showing promotion by salt-tolerant (halophilic) rhizobacteria isolated from halophyte species in saline soils (Mapelli et al., 2013; Rajput et al., 2013; Ruppel et al., 2013; Li et al., 2016; Orhan, 2016; Sharma et al., 2016; Kataoka et al., 2017; Palacio-Rodriguez et al., 2017; reviewed in Numan et al., 2018; Etesami and Beattie, 2018).

However, finding halophile bacteria that can be used to promote growth under saline conditions for saline-sensitive plants, and that can be used for a potentially commercial process has proven difficult. Halohilic bacteria can be found by isolating it from halophyte plant species. However, a screening process is required to determine the bacteria or combination of bacteria that have halophilic properties that benefit each species of plant. In Example 1 below, 40 samples had to be screened to identify 3 strains exemplified.

The bacteria, once screened, must be cultivatable. Even after screening and cultivation, a suitable symbiont bacteria may not have been found. Several screened and cultivatable bacteria have been found, but nonetheless, many will not promote growth of salt-sensitive plants under saline conditions. The bacteria have to form a symbiotic relationship with the new plant, a plant which is often a completely different plant than the natural host halophyte plant with which they evolved. Often there are specific factors or signals (molecules) that are required for establishing a relationship between the plant and bacteria, so there is no assurance that any new combination will work, and if it does work, whether its application only be valid for a small number of plants. For this reason, bacteria can be inoculated to a new non-host plant, but many predictably fail to form a symbiotic relationship.

In addition, to the bacteria identified herein and in the claims, several other isolates were tested for growth stimulation. Many did not stimulate growth of alfalfa in the presence of salt. The Bacillus GB03 strain was tested. This strain previously was shown to stimulate plant growth in the presence of salt (Xie et al., 2009; Han et al., 2014), but no noticeable growth stimulation of alfalfa in saline soil was observed. This suggests that the GB03 strain may enhance growth of only some plant species in the presence of salt. It is likely that different mechanisms are involved in each bacterial/plant interaction that leads to plant growth stimulation under saline conditions, and that a bacterial strain that stimulates growth of one plant species in the presence of salt may not cause similar stimulation in other plants.

Some examples include ginseng, where Paenibacillus yonginensis strain DCY84T protects against salinity stress by induction of defense related systems including ion transport, ROS enzyme production, proline content, total sugar and ABA biosynthesis related genes (Sukweenadhi et al., 2018). Another research group found that an endophytic strain of Bacillus amyloliquefaciens produces ABA in response to increasing salinity, increasing production of glutamic acid and proline to increase resistance to salinity in rice (Shahzad et al., 2017). In addition to these examples, there are multiple reports of different bacterial species that stimulate growth of a variety of plant species, supporting the notion that stimulation may be specific to the plant host and bacterial species (Bharti et al., 2016; Li et al., 2016; Mitter et al., 2013; Navarro-Torre et al., 2016; Yuan et al., 2016; also see the Introduction).

Halophytes are naturally salt-tolerant plants that have evolved to grow in saline soils; different halophyte species have different salt tolerance levels (Flowers and Colmer, 2015). As an example, much of the state of Utah is a high desert with saline soils, and a wide variety of halophytes are native to this area.

Studies have been made in understanding physiological mechanisms and gene expression changes involved in salt tolerance in halophytes (Shabala, 2013; Diray-Arce et al., 2015). Some halophytes have been developed or have potential for use as crop plants (Gul et al., 2014; Khan et al., 2009). Studies have also been made of microbes found in the rhizosphere (rhizobacteria) or within plant tissues including roots (endophytes), and whether these microbes have potential to contribute to the ability of plants to adapt to adverse conditions (Numan et al., 2018).

However, as described above, little is known about the potential contribution of microorganisms associated with halophyte plants in the soil, on plant surfaces, or within plant tissues of a non-host plant of an entirely different species, often of a separate genus or family of vascular plants.

Another aspect is an artificial salt tolerant plant and a method for forming same. It involves a glycophyte plant combined with a non-host halophile bacteria inoculated into the glycophyte plant rhizosphere or as an endophyte. This forms a non-natural or artificial plant having a symbiotic relationship with the non-host halophile bacteria to provide growth promotion to the plant under saline conditions and to form an artificial plant/bacteria combination as the salt-tolerant plant. The non-host halophile bacteria is identifiable as a naturally occurring soil bacteria associated with a halophyte plant that is a member of inland occurring (particularly in arid regions) halophyte plants of the subfamily Salicornioideae.

A further aspect is the characterization of previously unknown strains of soil bacteria that are associated with a certain family of inland halophytes, from the sub-family Salicornioideae, particularly from genus Allenrolfea, Salicornia, or Sarcocornia. These halophytes are found in saline environments, and identification/screening of the beneficial microorganisms for use as inoculants to stimulate growth of non-host plants under saline conditions were undertaken. It was found that inoculants isolated from three species of these halophytes showed an unexpected ability to combine symbiotically with a number of is glycophytes and form salt tolerant plants.

Another aspect is the discovery that certain isolates, for example, of the Halomonas and Bacillus genera, are able to be universal, i.e., form symbiotic relationships with most or a wide variety of non-host plants, including crop-plants, such those tested, alfalfa, Kentucky blue grass, and Bermuda grass, and cause growth stimulation. The results show bacteria used as inoculants to enhance growth of non-host plants under saline conditions. In the examples, three different species of non-host plants were evaluated. It is believed that the present teachings are applicable to any non-host plant, but particularly to any plant that is related to those tested, for example, related taxonomically, having overlapping evolutionary ancestry with similar gene sequences, from a similar environment, and the like. Accordingly, it is expected that the present teachings could be, for example, applied to a wide selection of grass species, particularly turf grass species like the exemplary Bermuda and Kentucky blue grasses.

As used in the claims and in this specification the following are defined as follows:

High salinity” or “saline” is defined as having a salt content or alkalinity sufficient to reduce yield 50% (based upon dry weight) or more of a salt-sensitive plant when compared to the same plant growing under non salty conditions. A rule of thumb for many plants is that an increase in salinity of about 0.5 weight percent will decrease the yield by about 50%.

In the examples, analysis of a saline site where the bacteria were isolated showed electrical conductivity (EC) of 18-70 dS/m, which corresponds to about 0.9-3.5% salt. Alfalfa fields just a few miles away have salinity of 0.5-1.6 dS/m (about 0.025-0.08% salt). These ranges depend on the time of year and amount of rainfall, which temporarily lower the salinity.

halophyte plant—a plant that can grow without material damage in high salinity environments;

glycophyte or glycophylic plant—non-halophyte plant or a natural plant that is salt-sensitive or not salt tolerant and is materially damaged by saline conditions. Growth yield is lowered progressively with increasing salinity, so it depends on the crop and conditions. Glycophytes, which include most crop plants, are affected by salinity levels around 5 dS/m (about 0.25% salt) or less, while halophytes grow well in 10-70 dS/m (about 0.5-3.5% salt), depending on the plant.

materially damaged or material damage—the damaged plant has a dry weight less that 50% than that of the same plant grown under non-saline conditions.

host halophile bacteria—halophile symbiotic bacteria existing either in the rhizosphere or an endophyte of a plant. These bacteria in nature exist with a host halophyte plant, and are referred to herein as “host halophile bacteria”.

non-host bacteria or non-host halophile bacteria—As applied according to the principles herein, halophile bacteria can exist with a plant in an artificial symbiotic relationship that does not exist in nature. The bacteria may be in the rhizosphere or be an endophyte of the plant, and the plant, therefore, is artificial in that it is not naturally occurring, co-existing with the non-host bacteria and being salt-tolerant.

artificial—As seen in the above definition, artificial means not natural or not occurring in nature.

inoculate or inoculum—the method and solution used to introduce non-host halophilic bacteria to a naturally occurring glycophyte plant and become associated with the plant in a symbiotic relationship.

In many cases a salt tolerant plant that has an increase in yield of at least 20-25% or more would be very helpful to farmers. In examples, increases of about 20-40% with alfalfa grown in salt after inoculation with the strains compared to uninoculated plants grown in the same salt conditions have been seen.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1. Collection site south of Utah Lake near Goshen, Utah. Panel A shows an overall view of the site. Panels B-D are close-up photos of each of the three halophyte species: B, Salicornia rubra; C, Sarcocornia utahensis; D, Allenrolfea occidentalis.

FIG. 2. Rhizosphere bacteria of the three halophytes in spring and fall samplings. Heat map and dendrogram showing relationships between the abundance of major bacterial families and samples collected in the fall and in the spring. ALOC, Allenrolfea occidentalis; SAUT, Sarcocornia utahensis; SARU, Salicornia rubra.

FIG. 3. Venn diagrams showing the distribution of shared and unique rhizobacterial species between the three halophyte species. Recovery was based on OTUs from bacterial community libraries of the 16S rRNA gene (97% similarity cutoff), with numbers indicated in each quadrant (not to scale). Abbreviations are as in FIG. 2.

FIG. 4. Growth stimulation of alfalfa seedlings in soil in the presence of 1% salt. Uninoculated control (LB media without bacteria), left. Inoculation with the Bacillus isolate, right.

FIG. 5. Box and whisker plot of stimulation of alfalfa growth by bacterial inoculation in the presence of 1% NaCl. Total mass is in milligrams. LB, control (no bacterial inoculation). Grown in the laboratory in replicate pots with three plants per pot.

FIG. 6A. Alfalfa growth stimulation by halophilic bacteria in salty soil. The photo shows 3 representative plants from each treatment.

FIG. 6B Alfalfa growth stimulation by halophilic bacteria in salty soil. Significant root length increase induced by the Halomonas (A07-1) and Bacillus (Su1-1) isolates.

FIG. 6C. Plant growth performance enhanced by halophilic bacteria. Each treatment had 30 plants, and plants were watered with 1% NaCl solution starting one week after bacterial inoculation and grown in the greenhouse.

FIG. 7A and FIG. 7B. Kentucky bluegrass samples grown in different conditions from each other. FIG. 7B is the same as 7A from above.

FIG. 8A and FIG. 8B. Harvested Kentucky bluegrass plant with adherent soil on roots (8A) and soil washed away (8B).

FIG. 9A, FIG. 9B, and FIG. 9C. Washed Kentucky bluegrass plants grown under different conditions from each other.

FIG. 10. Alfalfa plants modified with different inoculants from each other.

FIGS. 11A and 11B. Two views of Bermuda grass sample grown under different conditions from each other.

FIG. 12. Harvested Bermuda grass grown under different conditions from each other.

### DETAILED DESCRIPTION

The mechanisms by which halophilic bacteria stimulate plant growth include binding of salt ions by the bacteria or production of volatile compounds or other signals that stimulate expression of genes to enhance growth via increased photosynthesis or other changes in the host plant (Meena et al., 2017; Numan et al., 2018). Some microbes produce biofilms in the rhizosphere that trap water and nutrients and reduce plant uptake of sodium ions from the soil (Nadeem et al., 2014).

There are several mechanisms that may be involved in plant growth promotion by endophytes under non-saline conditions (Santoyo et al., 2016; Kim et al., 2012). Mechanisms by which endophytes enhance plant growth include acquisition of nutrients and altering expression of plant genes that affect growth and development. The endophyte Burkholderia phytofirmans PsJN enhances growth for six of the eight switchgrass cultivars that were tested (Kim et al., 2012). Inoculation with this strain was found to induce wide-spread changes in gene expression in the plant host, including transcription factors that are known to regulate expression of some plant stress factor genes (Lara-Chavez et al., 2015).

With respect to the non-host halophilic bacteria, it is believed that changes in plant gene expression are also induced by the non-host halophilic bacteria used to inoculate glycophyte plants, such as alfalfa, Kentucky blue grass, and Bermuda grass.

### Example 1

### Creation of Salt Tolerant Alfalfa

As described, halophytes are plants that have adapted to grow in saline soils, and have been widely studied for their physiological and molecular characteristics, but little is known about their associated microbiomes. Bacteria were isolated from the rhizosphere and as root endophytes of Salicornia rubra, Sarcocornia utahensis, and Allenrolfea occidentalis, three native Utah halophytes. A total of 41 independent isolates were identified by 16S rRNA gene sequencing analysis. Isolates were tested for maximum salt tolerance, and some were able to grow in the presence of up to 23.4% NaCl. For comparison, ocean water is about 3.5% salt. The salt level where the bacteria were collected ranged from about 1.46 to 1.64%. The salt is mostly but not all in the form of NaCl as there are other salts present in the soil. Alfalfa growth is affected by as little as 0.5% NaCl or less. The more salt, the more growth is diminished.

Pigmentation, Gram stain characteristics, optimal temperature for growth, and biofilm formation of each isolate aided in species identification. Some variation in the bacterial population was observed in samples collected at different times of the year, while most of the genera were present regardless of the sampling time. Halomonas, Bacillus and Kushneria species were consistently isolated both from the soil and as endophytes from roots of all three plant species at all collection times. Non-culturable bacterial species were analyzed by Illumina DNA sequencing. The most commonly identified bacteria were from several phyla commonly found in soil or extreme environments: Acidobacteria, Actinobacteria, Bacteroidetes, Chloroflexi, and Gamma- and Delta-Proteobacteria. Isolates were tested for the ability to stimulate growth of alfalfa under saline conditions. This screening led to the identification of one Halomonas, one Kushneria, and one Bacillus isolate that, when used to inoculate young alfalfa seedlings, stimulate plant growth in the presence of 1% NaCl, a level that significantly inhibits growth of uninoculated plants. The same bacteria used in the inoculation were recovered from surface sterilized alfalfa roots, indicating the ability of the inoculum to become established as an endophyte. The results with these isolates indicate that enhanced growth of inoculated alfalfa in salty soil can be achieved.

Little previous work has been published on microbiomes associated with native halophytes in desert areas of the United States. Halophilic microbes in and near the Great Salt Lake and other marine environments have been studied, but aquatic species are different from those found in desert soil.

This example focuses on the microbiomes of three halophyte species that grow in a highly saline area south of Utah Lake where soil salinity is between 16 and 100 dS/m (compared to local land where alfalfa is growing that is 0.7-1.6 dS/m and ocean water which is about 55 dS/m). DNA sequence analysis of the isolates identified species of a number of known halophilic genera. Some isolates are capable of growth in up to 4 M NaCl, and two isolates show promise for use as inocula for alfalfa to stimulate growth in salty soil.

### Materials and Methods

Collection of Samples

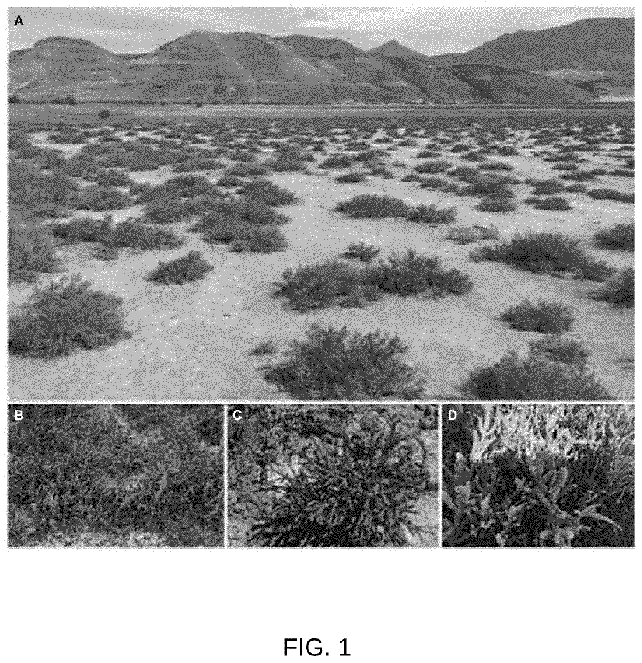

Six collection trips were made to a study site near Goshen, Utah (coordinates: 39:57:06N 111:54:03W, 1360 m above sea level; Gul et al., 2009) (FIG. 1). At this study site there are three predominant halophyte species, each native to Utah (Salicornia rubra, Sarcocornia utahensis, and Allenrolfea occidentalis; all are members of the same subfamily, Salicornioideae). Individual plants of each of the three species were removed from the ground, and samples of soil adhering to the roots and root tissue were separately collected into sterile tubes for transport to the lab. Disposable gloves were worn for each sample to avoid cross-contamination between samples and from human-associated microbes. Soil was also collected from bare areas where no plants were growing for comparison. Soil was analyzed by the BYU Soils Lab for salinity level and pH. Soil salinity was measured using a Beckman RC-16C conductivity bridge to measure electrical conductivity as dS/m. Soluble salts and pH were measured in saturated soil pastes. Soil samples were mixed with deionized water, the saturated mix was allowed to sit overnight for the soil to settle, and the pH of the liquid was measured with a standard pH meter.

Isolation and Characterization of Bacteria

Rhizosphere soil samples were vortexed in buffer (0.5 g sample in 1 ml 1×PBS (phosphate buffered saline) and plated on LB (Luria broth) agar plates containing 1 M NaCl. To isolate endophytic bacteria, root samples were surface sterilized (by washing twice in sterile distilled water, once for 10 min in 70% ethanol, and twice in sterile PBS) and ground in PBS buffer. Cultures were re-streaked on LB media containing increasing amounts of NaCl (1M, 2M, 3M, 4M) to determine maximum salt tolerance of each isolate. Bacterial isolates were also tested for maximum salt tolerance on M9 minimal salts media agar plates. Colony morphology, pigmentation, and the temperature range of growth for each isolate were also determined. Individual colonies were used to inoculate liquid LB+0.25 M NaCl and incubated overnight with shaking at 30° C. Stock cultures of each isolate were stored at −80° C. in 20% glycerol.

Bacterial Identification

To identify the bacteria, genomic DNA was obtained from individual isolates using a DNA isolation protocol that involves lysis and digestion of nucleases with proteinase K (Chachaty and Saulnier, 2000). For each sample the 16S ribosomal RNA (rRNA) gene was amplified by PCR using the 8F and 1492R primers (Turner et al., 1999) for sequence determination at the Brigham Young University Sequencing Center (http://dnac.byu.edu/; Sanger sequencing protocol). Sequences obtained were used to identify the genus and species by BLAST search of the NIH/NCBI bacterial database. Forty one individual sequences were submitted to Gen Bank, accession numbers MK873873-MK873913. Colony morphology and Gram staining were utilized to assist in identification of the species (Vreeland et al., 1980; Zhang et al., 2007).

To identify nonculturable bacteria in the halophyte rhizosphere samples, bacterial communities were characterized on roots using barcoded next-generation sequencing of the 16S rRNA gene in a metagenomic approach. Genomic DNA were extracted from 1.0 g of rhizosphere soil using the DNeasy Powersoil Kit (Qiagen Inc., Germantown, Md., USA). The V4 region of the 16S rRNA gene was amplified using the bacterial specific primer set 515F and 806R with unique 12 nt error correcting Golay barcodes (Aanderud et al., 2016). Barcoded samples were purified (Agencourt AMPure XP PCR Purification Beckman Coulter Inc., Brea, Calif., USA) and normalized with a SequalPrep Normalization Plate Kit (invitrogen, Carlsbad, Calif., USA); pooled at approximately equimolar concentrations after being quantified with an Agilent 2100 Bioanalyzer (Agilent, Santa Clara, Calif., USA). All samples were sequenced at the Brigham Young University DNA Sequencing Center (http://dnasc.byu.edu/) via two 250 bp paired-end sequencing on an illumina HiSeq 2500 System (HiSeq Rapid SBS Kit v2, illumine, San Diego, Calif., USA). All sequences were processed using the mothur (v. 1.39.0) pipeline https://www.mothur.org/wiki/MiSeq_SOP; Schloss et al., 2009; Kozich et al., 2013). After removing barcodes and primers, sequences were eliminated that were <250 bp in length or sequences possessing homopolymers longer than 8 bp. The sequences were denoised with AmpliconNoise (Quince et al., 2011), removed chimeras with UCHIME (Edgar et al., 2011), and eliminated chloroplast, mitochondrial, archaeal, and eukaryotic gene sequences based on reference sequences from the Ribosomal Database Project (Cole et al., 2009). Sequences were aligned against the SILVA database (silva.nr_v132, Pruesse et al., 2007) with the SEED aligner to create operational taxonomic units (OTUs) based on uncorrected pairwise distances at 97% sequence similarity. Phylogenetic identity of the OTUs was determined with the SILVA database and all samples were rarified to a common sequence number (29,000). Multivariate statistics on the rhizosphere communities were performed in R (R Development Core Team, 2018). Specifically, the phylogenetic trends of 39 dominant bacterial families (mean recovery ≥0.05% in any sample) from 11 phyla were represented in a heat map with hierarchal clustering using the heatmap function in the ‘gplot’ package (Oksanen et al., 2013). Venn diagrams created with the ‘venneuler’ package were used to examine differences between OTUs in the different rhizosphere samples. The Illumine sequence reads are available at the NCBI Sequence Archive under BioProject ID PRJNA553550, BioSample accessions SAMN12238110, SAMN12238111, SAMN12238112, SAMN12238113, SAMN12238114, SAMN12238115, SAMN12238116, SAMN12238117, SAMN12238118, SAMN12238119 (https://www.ncbi.nlm.nih.gov/Traces/@study/?acc=PRJNA553550.

Analysis of Biofilm Formation of Isolates

Bacterial isolates were tested for the ability to form biofilms in 96 well plates, generally following published protocols (Coffey and Anderson, 2014) with minor modifications. Briefly, overnight liquid cultures were diluted to an OD₆₀₀ of 0.4, and 100 μl was seeded into each well of a 96 well plate. Each culture was seeded in triplicate in random locations in the plate to avoid position effects. The plate was sealed and incubated at 30° C. for 24 hours (without any shaking). The liquid media was then carefully removed and the wells were stained with 100 μl of 0.01% crystal violet for 20 min at room temperature. The stain was then removed, wells were washed twice with sterile distilled water, and the remaining dye in each well was solubilized by adding 100 μl of 30% acetic acid and pipetting up and down to fully suspend and mix the dye. The plate was scanned at OD₅₇₀ to measure biofilm levels for each sample.

Plant Growth Stimulation Trials with Microbiome Isolates

Individual isolates were evaluated for the ability to stimulate growth of young alfalfa seedlings when used as an inoculum. These initial trials were done with autoclaved soil and sterilized seeds in closed pots (see details below) to remove any bacteria from the soil and on or within the seeds, to ensure that the only bacteria present would be the inoculum (except for the uninoculated controls). Alfalfa seeds were sterilized with dilute bleach (1% sodium hypochlorite) for 10 min, followed by two washes with sterile water and incubation for 1 hour in 70% ethanol, followed by four washes with sterile water (all steps at room temperature). The seeds were then allowed to germinate in a sterile petri dish in a small amount of water. After 36-48 hours, the seedlings were transplanted into autoclaved soil (1:1:1 Miracle Grow potting soil (miraclegro.com):clay:sand) in a clear magenta box. One hundred ml of 0.5× Hoagland's basic nutrient solution containing 0, 0.5% or 1% NaCl (or as indicated if otherwise) along with 1 ml of the bacterial culture to be tested as inoculum was added to each box. Bacillus strain GB03 was obtained from the Bacillus Genetics Stock Center (bgsc.org, stock ID 3A37) and also tested for growth promotion of alfalfa in the presence of salt. Similar samples without bacteria (sterile LB broth only) were included as experimental controls. Three seedlings were transplanted into each box, repeated for a total of six replicates (two boxes per inoculum or control for a total of 6 plants per treatment). For each replicate box a second magenta box was inverted and taped in place with a small gap (˜2 mm) on one side to allow for air exchange while reducing evaporation. Boxes were placed in a plant growth room with a 16 hr light (@82 mmol m⁻² s⁻¹)/8 hr dark cycle at 22° C. and ambient humidity with no further watering. After 6 weeks of growth, plant height and total weight and length of shoots and roots were measured. Uninoculated plants were included as controls. After confirming normality of the data, differences in shoot and root length among inoculated and control plants were determined using one-way ANOVA with a Tukey's HSD test using R.

To confirm the presence of the bacterial inoculum at the conclusion of the growth experiment, soil and root samples were collected when the plants were harvested. Soil was diluted in sterile PBS and spread on LB agar plates containing 1 M NaCl as before. Roots were surface sterilized, ground in sterile PBS, and similarly spread on plates. DNA was isolated from colonies and sequenced as before, and colony morphology was compared to confirm that the recovered bacteria were the same as those used to inoculate the plants.

Greenhouse Trials

The next step was to test the bacterial isolates in open pots in the greenhouse. For this, alfalfa seeds were surface-sterilized with 50% Chlorox® bleach for 10 min, rinsed with sterile water 5 times, and germinated in an incubator for 2 days. Three seedlings were transplanted into open pots (15 cm round) containing Miracle-Gro® Potting mix (miraclegro.com) and grown in the greenhouse under natural light with temperatures at 25±2.0° C./day time and at 18±2.0° C./night time, and humidity with 45-70%. On the following day each seedling was inoculated with 1 ml of halophilic bacteria at 1.0 of OD₆₀₀ suspended in PBS buffer. Control uninoculated seedlings were supplemented with 1 ml of PBS buffer. Each treatment had 10 pots. Salt treatment started 7 days after halophilic bacterial inoculation with 1% NaCl solution. Plants were harvested one month after salt treatment. Soil was washed out with tap water, and lengths and fresh weights of shoots and roots were measured. Data analysis was conducted with one-way ANOVA and LSD comparison using SAS University Edition.

### Results

Recovery and Characterization of Rhizospheric and Endophytic Bacteria

The collection site primarily consists of highly saline soil with three dominant halophyte species, Allenrolfea occidentalis, Salicornia rubra, and Sarcocornia utahensis (FIG. 1). This site is just south of Utah Lake with high salinity due to the evaporation of water since the collapse of ancient Lake Bonneville more than 14,000 years ago (Weber, 2016). This area is about 1.5 miles away from productive alfalfa fields where soil is much less saline (0.7-1.6 dS/m compared to 16-100 dS/m where the halophyte samples were collected). Soil salinity around the plants ranged from 16-18 dS/m in the spring, and up to 70 dS/m in the fall (Table 1). This variation is likely due to the majority of rainfall occurring during the winter and early spring months followed by very dry summers. In areas where no plants were growing salinity was between 45 and 100 dS/m depending on the season. All soil samples had a pH between 7.56 and 7.98 (Table 1).

Bacterial isolates were recovered from the rhizosphere samples on LB agar plates containing 1 M NaCl. Isolates were found to have varying levels of maximum salt concentration tolerance for growth, with some growing in the presence of up to 4 M NaCl (Table 2). The isolates grew equally well on minimal media agar plates at the same salt concentrations. The temperature range for growth, pigmentation, and colony morphology were recorded for each isolate (Table 2). Colony morphology aided in identification of genus (Vreeland et al., 1980; Zhang et al., 2007). For example, Kushneria forms bright red-orange colonies (Sanchez-Porro et al., 2009).

DNA Sequence Analysis and Bacterial Species Identification

BLAST analysis of the 16S rRNA amplicon sequences from 41 independent isolates was performed to identify the bacteria recovered (details are available for each via the GenBank accession numbers that are included in Materials and Methods for all isolates and in Table 2 for selected isolates). Many of the isolates were identified from the same genus and could not be further identified at the species level based on colony morphology or Gram stain. The most common bacterial genera recovered were Halomonas (16 of the 41 isolates tested), Bacillus (16 isolates), and Virgibacillus (4 isolates). There were two isolates from Kushneria and one isolate each from Oceanobacillus, Vibrio and Zhihengiluella.

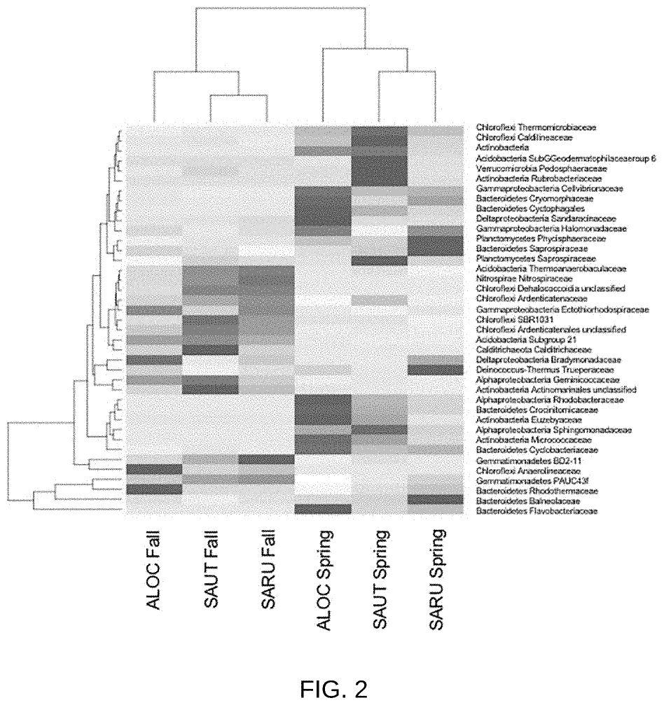

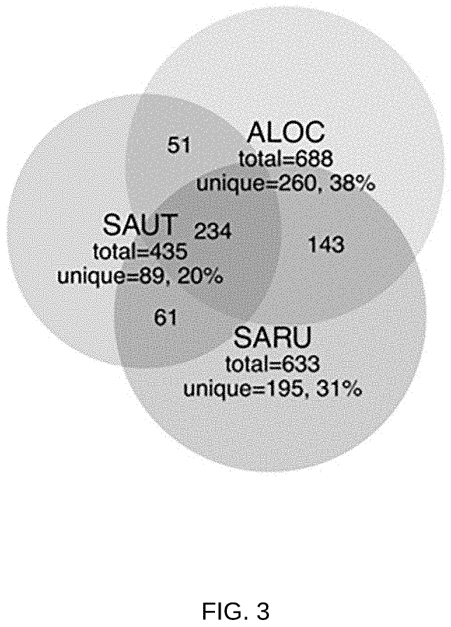

To obtain a more detailed picture of total bacterial diversity associated with each halophyte, total rhizosphere DNA was analyzed by Illumina sequencing. Next-generation sequencing of the 16S rRNA gene (shown in FIGS. 2 and 3) identified some similar OTUs as the plated isolates. For example, Halomonas, Kuchneria, Bacillus and several others were identified by both approaches. Further, bacterial communities were sensitive to seasonal fluctuation from the spring to fall (FIG. 2). This is likely at least partially due to the significant difference in soil salinity, increasing from 16-18 dS/m in the spring to about 70 dS/m in the fall when samples were collected from the halophytes, while soil pH remained about the same. Based on unique OTUs in rhizospheres, bacterial communities were more unique on roots of Allenrolfea occidentalis than Salicornia rubra and Sarcocornia utahensis (FIG. 3). For example, the number of unique OTUs in Allenrolfea occidentalis rhizospheres was at least 1.3-times higher than the Salicornia species.

Species or OTUs identified were from the Cryomorphaceae, Cytoophagales, Flavobacteriaceae, Rhodothermaceae (Bacteriodetes) and Anaerolineaceae (Chloroflexi). Bacterial community results were based on the recovery of 175,239 quality sequences and 3,550 unique OTUs with samples possessing an average sequencing coverage of 97%±0.003 (mean±SEM).

Characterization of Isolates for Biofilm Formation

The Halomonas, Kushneria and Zhihengliella isolates form biofilms when grown in LB+0.25 M NaCl, while the other isolates tested do not form or poorly form detectable biofilm (summarized in Table 2). Biofilm formation by some bacterial strains has been shown to be associated with soil adherence to plant roots in some studies (Qurashi and Sabri, 2012).

Screening of Isolates for Alfalfa Growth Stimulation Capabilities

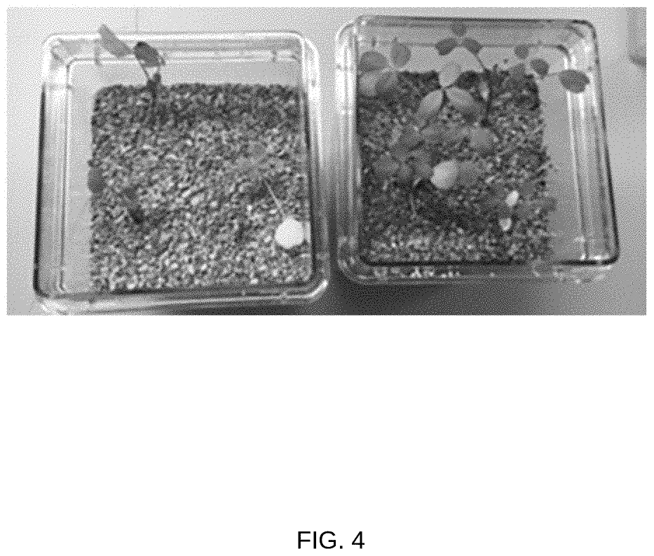

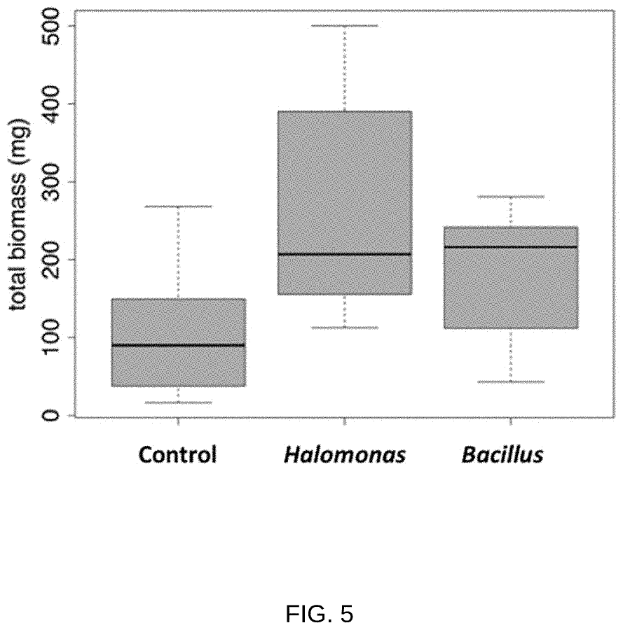

The salt-tolerant bacterial isolates were tested for the ability to stimulate growth of alfalfa under saline conditions. This screening identified Halomonas (MK873884) and Bacillus (MK873882) isolates that significantly stimulated growth when used to inoculate alfalfa (FIGS. 4, 5). Total biomass was 2.4-times higher in alfalfa inoculated with Halomonas than uninoculated alfalfa (one-way ANOVA, F=3.1, P=0.06, df=2). While this has only borderline significance, similar results were obtained with repeated trials. Some other isolates appeared to inhibit or to have little effect on plant growth. A few strains had a slight stimulatory effect on plant growth, including some Pseudomonas species, Kushneria, Bacillus subtilis strain GB03, Bacillus licheniformis and some mixed cultures (not shown).

Recovery of Inoculum from Soil and Roots of Inoculated Plants

To determine whether the bacterial inoculum was able to colonize the soil and/or become endophytic in alfalfa roots, soil and root samples were collected when the alfalfa plants were harvested and plated as before. Colonies showed the same characteristics as the bacteria used to inoculate the plants, and DNA was isolated and sequenced to confirm identity (Halomonas (MK873884) and Bacillus (MK873882)). Roots from plants inoculated with these two isolates also yielded the same bacteria (ranging from 3000-8000 colonies per gram of soil) used to inoculate the plants, while the control LB plants and those inoculated with one of the other Bacillus isolates did not yield bacteria.

The observation that the Halomonas and Bacillus isolates were able to form endophytic relationships with alfalfa leading to growth stimulation shows their potential use as inoculants to enhance growth of non-host plants under saline conditions.

Growth Stimulation in Greenhouse Studies

The initial growth stimulation trials were performed in closed pots in a controlled environment. It was desired to scale up the experiments in greenhouse trials at the Institute for Advanced Learning and Research. Alfalfa plants were grown in open pots with carefully controlled watering and growth monitoring. As with the earlier studies, plants were grown with and without inoculation with the Halomonas and Bacillus isolates, in the presence and absence of 1% NaCl in the watering solution. In the absence of salt in the watering solution there were no differences in either shoot or root biomass between halophilic bacterial inoculation and control treatment.

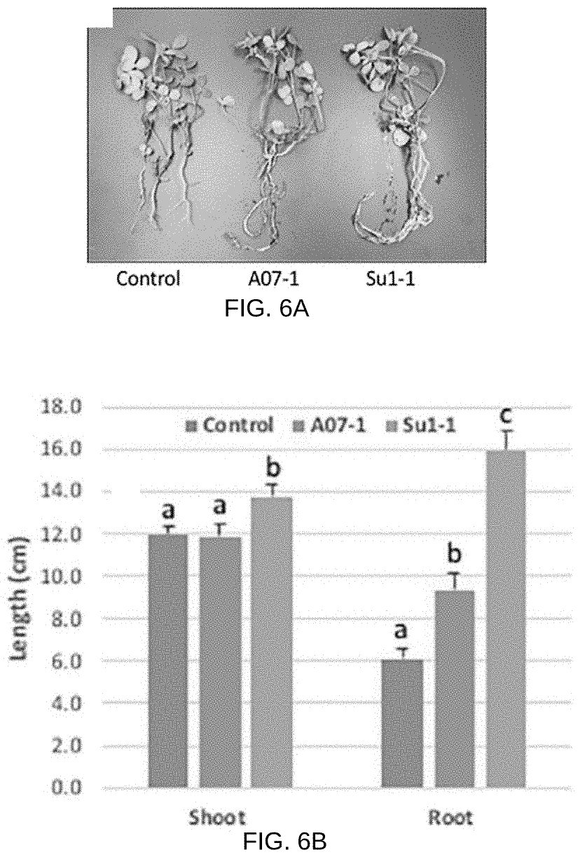

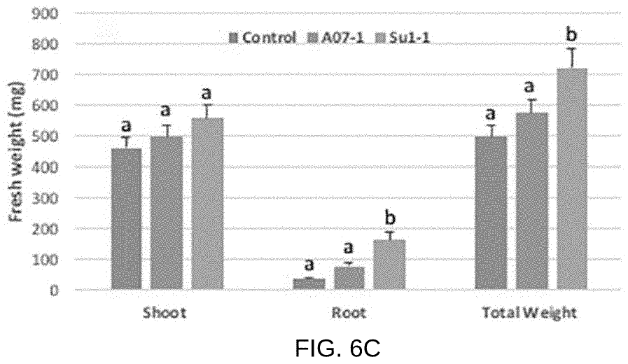

As shown in FIGS. 6A, 6B, and 6C, the inoculation of both Halomonas and Bacillus isolates stimulated alfalfa root growth, with root length increasing 2.6-fold in Halomonas and 1.5-fold in Bacillus inoculated plants relative to uninoculated control alfalfa (one-way ANOVA, F=43.85, P<0.0001, df=2). Shoot length was also elevated but only for Bacillus (one-way ANOVA, F=3.23, P=0.0444, df=2). In addition, Bacillus (Su1-1) showed much better performance than Halomonas (A07-1) in root and shoot biomass, with at least 4.5-fold increase in root fresh weight over the control treatment (one-way ANOVA, F=14.45, P<0.0001, df=2) and only 21% increase in shoot fresh weight over the control treatment (one-way ANOVA, F=1.28, P>0.2848, df=2). Total fresh weight was significantly increased by Bacillus (Su1-1) (one-way ANOVA, F=4.92, P<0.0095, df=2). In addition, both the Halomonas inoculation and uninoculated control treatments had 2 dead plants while the Bacillus inoculation treatment had no dead plants.

### Discussion

Production of sufficient food for the world's population is a critical challenge, exacerbated by the loss of agricultural land to urbanization, degradation of existing land, diminished water quality, and salinization of soil in many areas. These factors leave farmers in many parts of the world with access only to poor land (low soil quality) and/or poor water quality to produce crops for human consumption and for animal feed. The development of crop plants that are able to adapt and grow sustainably under changing environmental stresses is of urgent importance.

Our objective in this example was to make a general survey of the types of bacteria that are present in association with three species of halophytes in central Utah (Salicornia rubra, Sarcocornia utahensis, and Allenrolfea occidentalis). 41 isolates were identified, including multiples from the same genus, of culturable halophilic bacteria. These strains vary in their ability to form biofilms, in the maximum concentration of salt that allows growth, and in pigmentation and colony morphology. Several of the isolates had strong yellow, orange or red pigmentation due to carotenoids that may help protect the bacteria from damaging UV radiation (Khaneja et al., 2010). Halomonas species (based on sequencing and colony morphology they are most likely H. elongate or H. huangheensis) were found as root endophytes and in the rhizosphere of all three halophytes. Halomonas and Kushneria are closely related, and in the past were grouped in the same genus (Sanchez-Porro et al., 2009). Analysis of total soil or root tissue identified many other non-culturable bacteria, including members of common soil phyla and some that are present in extreme environments such as desert and saline conditions. The rhizosphere of Allenrolfea occidentalis supported the highest number of unique OTUs (260 OTUs or 38% of OTUs), while Sarcocornia utahensis supported the lowest number of unique species (89 OTUs or 20% of OTUs). At least 34% of rhizosphere OTUs were shared among the three species.

A very important advance resulting from the screening of isolates for plant growth promotion capabilities was the identification of two that support growth of alfalfa in saline soil when used to inoculate young seedlings. When used to inoculate alfalfa seedlings, Halomonas and Bacillus stimulated alfalfa growth in soil watered with 1% NaCl, with Bacillus showing the greater stimulation of growth of both shoots and roots. Bacteria recovered from roots of inoculated alfalfa were the same as used for the inoculation, indicating that these strains may be useful for inoculation of alfalfa to enhance plant growth in salty soil.

### Example 2

Bacteria strains B1—Bacillus, B2—Kuchneria, and B3—Halomonas were prepared essentially the same as in Example 1, and were used in the examples below. Plants grown in salt were grown in 1% NaCl in Hoagland's Solution.

The following show growth comparisons between inoculated plants in salt, and non-inoculated plants in salt and without salt. These demonstrate the unexpected salt tolerance of plants created by inoculation with certain bacteria strains.

### Kentucky Blue Grass

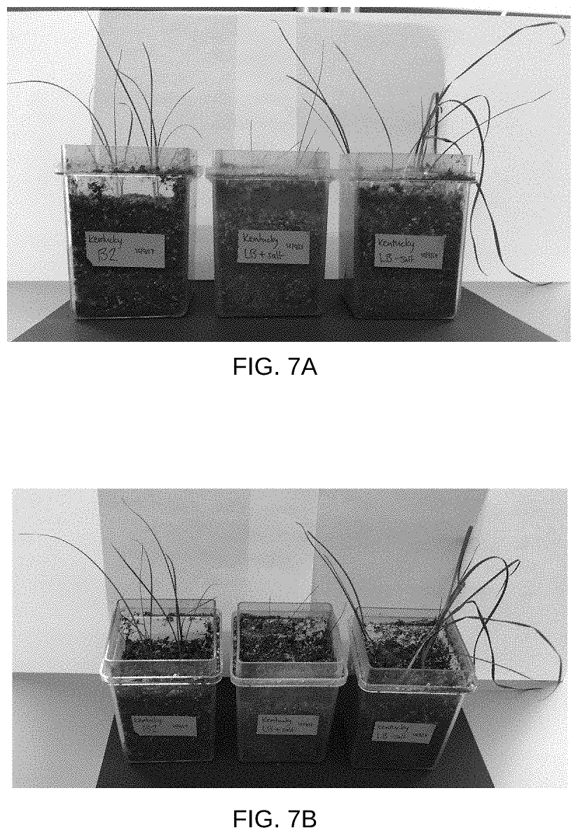

Three samples of Kentucky bluegrass were grown and are shown in FIGS. 7A and 7B from left to right: The inoculum was strain B2

(1) grown in 1% NaCl in Hoagland's Solution and inoculated with strain B2,
(2) control grown in 1% NaCl in Hoagland's Solution, and
(3) control grown without salt.
Artificial salt tolerant plants (1) showed 5.5× (fresh weight) growth compared to salt control (2). This compared with (3) control without salt, which had 8.4× growth compared to salt control (2).

### Kentucky Blue Grass

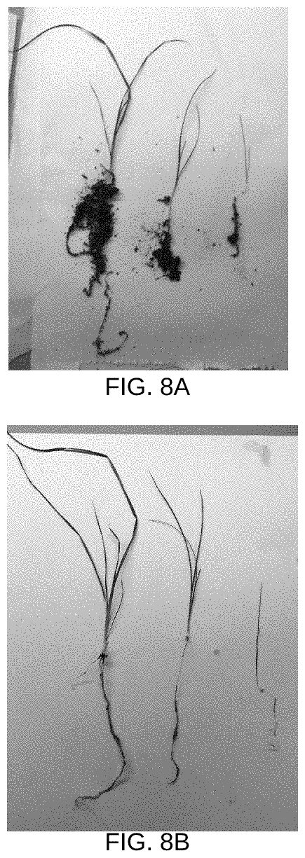

Shown in FIGS. 8A and 8B are harvested Kentucky bluegrass plants with adherent soil on roots (8A) and with soil washed away (8B).

In each figure, uninoculated plants grown in absence of salt (left), plants inoculated with strain B2 and grown in presence of salt (middle), and uninoculated plants in presence of salt (right).

### Kentucky Blue Grass

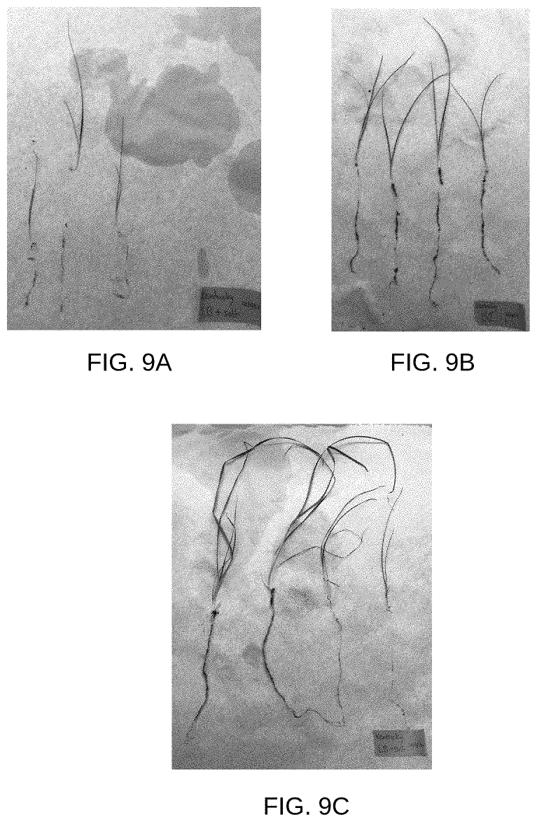

FIGS. 9A, 9B and 9C show washed Kentucky bluegrass plants, as follows:

FIG. 9A. Uninoculated plants in the presence of salt,
FIG. 9B plants inoculated with strain B1 in salt, and
FIG. 9C uninoculated plants grown in absence of salt.

### Alfalfa

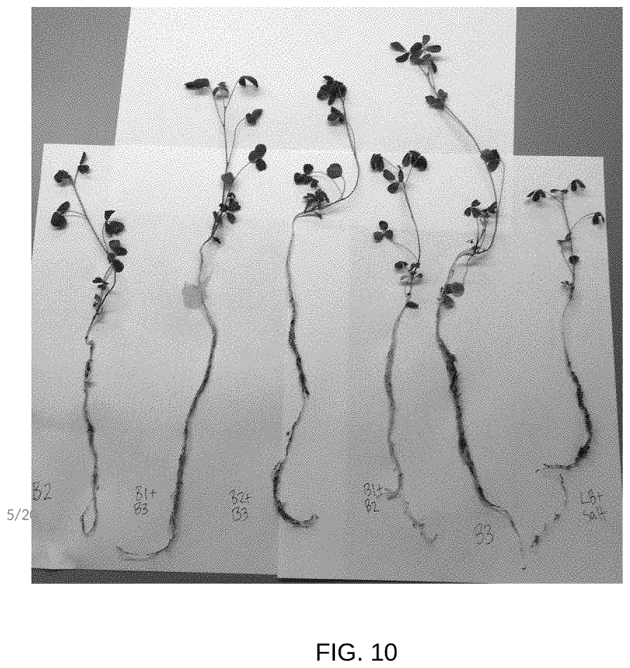

FIG. 10 shows alfalfa grown in salt after inoculation with various bacteria strains and combinations, from left to right; B2, B1+B3, B2+B3, B1+B3, B3 and plant with no inoculation grown in salt. The greatest growth stimulation was shown by strain B3 (6.1× increase in total fresh weight; 2.1× increase in dry weight), and combination B2+B3 (2.6× increase in fresh weight; 1.2× increase in dry weight).

### Bermuda Grass

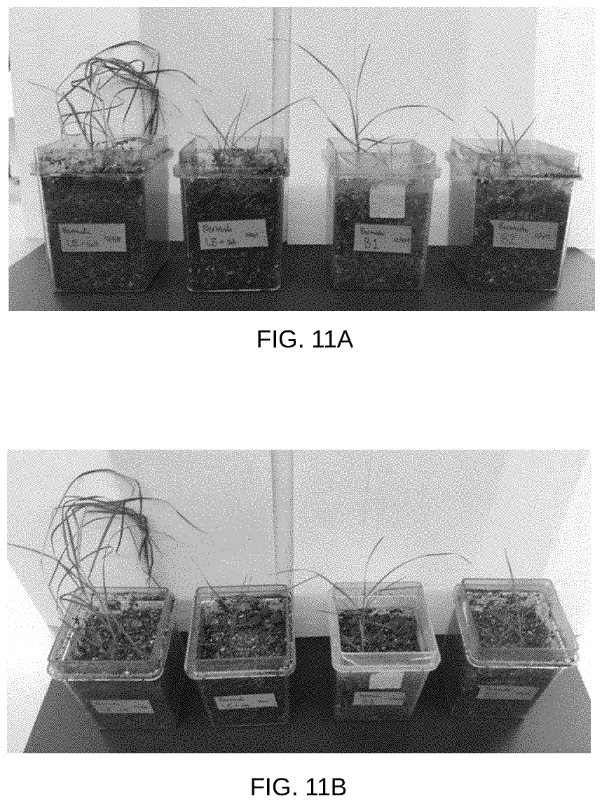

FIGS. 11A and 11B show different views of Bermuda grass samples grown as follows, from left to right;

(1) uninoculated plant grown without salt,
(2) uninoculated plant grown with salt,
(3) plant inoculated with B1 and grown in salt,
(4) plant inoculated with B2 and grown in salt.

Bermuda grass-appears to have more salt tolerance in the absence of bacterial inoculation. Strain B1 (3) shows greatest stimulation in salt (1.7× increase in total fresh weight compared to the uninoculated control (2)). Strain B2 does not appear to stimulate growth compared to the control.

### Bermuda Grass

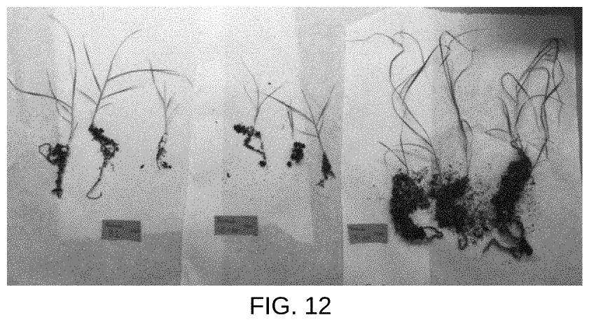

FIG. 12 shows Bermuda grass after harvesting. Inoculated with strain B1 and grown in salt (left), uninoculated plants in salt (middle), and uninoculated plants grown without salt (right).

The invention has been described with reference to various specific and preferred embodiments and techniques. Nevertheless, it is understood that many variations and modifications may be made while remaining within the spirit and scope of the invention.

TABLE OF REFERENCES

Aanderud, Z. T., Vert, J. C., Lennon, J. T., Magnusson, T. W.,
Breakwell, D. P., and Harker, A. R. 2016. Bacterial dormancy is more
prevalent in freshwater than hypersaline lakes. Front. Microbiol. 7, 853.
doi: 10.3389/fmicb.2016.00853
Bharti, N., Pandey, S. S., Barnawal, D., Patel, V. K., and Kalra, A.
(2016). Plant growth promoting rhizobacteria Dietzia natronolimnaea
modulates the expression of stress responsive genes providing protection
of wheat from salinity stress. Scientific Rep. 6, 34768. doi: 10.1038/
srep34768
Chachaty, E., and Saulnier, P. (2000) Bacterial DNA extraction for
polymerase chain reaction and pulsed-field gel electrophoresis, pgs.
33-36. In: Rapley, R. (ed.) The Nucleic Acid Protocols Handbook.
Humana Press, Totowa, NJ. dot: 10.1385/1-59259-038-1: 33
Coffey, B. M., and Anderson, G. G. (2014). Biofilm formation in the
96-well microtiter plate. In Pseudomonas Methods and Protocols,
Meth. Mol. Biol. 1149, 631-641.
Cole, J. R., Wang, Q., Cardenas, E., Fish, J., Chai, B., Farris, R. J., et al.
(2009). The Ribosomal Database Project: Improved alignments and new
tools for rRNA analysis. Nucleic Acids Res. 37, D141-D145. doi:
10.1093/nar/gkn879
Diray-Arce, J., Gul, B., Khan, M. A., and Nielsen, B. L. (2015).
Halophyte transcriptomics: understanding mechanisms of salinity
tolerance. In Proceedings of the International Conference on Halophytes
for Food Security in Dry Lands. Elsevier. doi: 10.1016/B978-0-12-
801854-5.00010-8
Edgar, R. C., Haas, B. J., Clemente, J. C., Quince, C., Knight, R. (2011).
UCHIME improves sensitivity and speed of chimera detection.
Bioinformatics 27, 2194-2200. doi: 10.1093/bioinformatics/btr381
Etesami, H., and Beattie, G. A. (2018). Mining halophytes for plant
growth-promoting salt tolerant bacteria to enhance the salinity tolerance
of non-halophytic crops. Front. Microbiol. 9, 148. doi: 10.3389/fmicb.
2018.00148
Flowers, T. J., and Colmer, T. D. (2015). Plant salt tolerance:
adaptations in halophytes. Annals Bot. 115, 327-331.
Gul, B., Ansari, R., and Khan, M. A. (2009). Salt tolerance of Salicornia
utahensis from the Great Basin Desert. Pakistan J. Bot. 41, 2925-2932.
Gul, B., Ansari, R., Ali, H., Adnan, M. Y., Weber, D. J., Nielsen, B. L.,
Koyro, H. W., and Khan, M. A. (2014). The sustainable utilization of
saline resources for livestock feed production in arid and semi-arid
regions: a model from Pakistan. Emirates J. Food Agricul. 26,
1032-1045.
Han, Q. Q., Lu, X. P., Bai, J. P. Qiao, Y., Pare, P. W., Wang, S. M.,
Zhang, J. L., Wu, Y. N., Pang, X. P., Xu, W. B., and Wang, Z. L. (2014).
Beneficial soil bacterium Bacillus subtilis (GB03) augments salt
tolerance of white clover. Front. Plant Sci. 5, 525. doi: 10.3389/fpls.
2014.00525
Kataoka, R., Guneri, E., Turgay, O. C., Yaprak, A. E., Sevilir, B., and
Baskose, I. (2017). Sodium-resistant plant growth-promoting
rhizobacteria isolated from a halophyte Salsola grandis, in saline-
alkaline soils of Turkey. Eurasian J. Soil Sci. 6, 216-225.
Khan, M. A., Ansari, R., Ali, H., Gul, B., and Nielsen, B. L. (2009).
Panicum turgidum, a potentially sustainable cattle feed alternative to
maize for saline areas. Agricul. Ecosyst. Environ. 129, 542-546.
Khaneja, R., Perez-Fons, L., Fakhry, S., Baccigalupi, L., Steiger, S., To,
E., Sandmann, G., Dong, T. C., Ricca, E., Fraser, P. D., and Cutting,
S. M. (2010). Carotenoids found in Bacillus. J. Appl. Microbiol. 108,
1889-1902. doi: 10.1111/j.1365-2672.2009.04590.x
Kim, S., Lowman, S., Hou, G., Nowak, J., Flinn, B., and Mei, C. (2012).
Growth promotion and colonization of switchgrass (Panicum virgatum)
cv. Alamo by bacterial endophyte Burkholderia phytofirmans strain
PsJN. Biotechnol. Biofuels 5, 37.
Kozich, J. J., Westcott, S. L., Baxter, N. T., Highlander, S. K., and
Schloss, P. D. (2013). Development of a dual-index sequencing strategy
and curation pipeline for analyzing amplicon sequence data on the
MiSeq Illumina sequencing platform. Appl. Environ. Microbiol. 79,
5112-5120.
Lara-Chavez, A., Lowman, S., Kim, S., Tang, Y., Zhang, J., Udvardi,
M., Nowak, J., Flinn, B., and Mei, C. 2015. Global gene expression
profiling of two switchgrass cultivars following inoculation with
Burkholderia phytofirmans strain PsJN. J. Exp. Bot. 66, 4337-4350.
Li, X., Geng, X., Xie, R., Fu, L., Jiang, J., Gao, L., and Sun, J. (2016).
The endophytic bacteria isolated from elephant grass (Pennisetum
purpureum Schumach) promote plant growth and enhance salt tolerance
of Hybrid Pennisetum. Biotechnol. Biofuels 9, 190. doi: 10.1186/s13068-
016-0592-0
Mapelli, F., Marasco, R., Rolli, E., Barbato, M., Cherif, H., Guesmi, A.,
Ouzari, I., Daffonchio, D., and Bonin, S. (2013). Potential for plant
growth promotion of Rhizobacteria associated with Salicornia growing
in Tunisian hypersaline soils. Biomed. Research Intl. 2013, 248078.
doi: 10.1155/2013/248078
Meena, K. K., Sorty, A. M., Bitla, U. M., Choudhary, K., Gupta, P.,
Pareek, A., Singh, D. P., Prabha, R., Sahu, P. K., Gupta, V. K., Singh,
H. B., Krishanani, K. K., and Minhas, P. S. (2017). Abiotic stress
responses and microbe-mediated mitigation in plants: the Omics
strategies. Front. Plant Sci. 8, 172. doi: 10.3389/fpls.2017.00172
Mitter, B., Petric, A., Shin, M. W., Chain, P. S. G., Hauberg-Lotte, L.,
Reinhold-Hurek, B., Nowak, J., and Sessitsch, A. (2013). Comparative
genome analysis of Burkholderia phytofirmans PsJN reveals a wide
spectrum of endophytic lifestyles based on interaction strategies with
host plants. Front. Plant Sci. 4, 120. doi: 10.3389/fpls.2013.00120
Nadeem, S. M., Ahmad, M., Zahir, Z. A., Javaid, A., and Ashraf, M.
(2014). The role of mycorrhizae and plant growth promoting
rhizobacteria (PGPR) in improving crop productivity under stressful
environments. Biotechnol. Adv. 32, 429-448.
Navarro-Torre, S., Barcia-Piedras, J. M., Mateos-Naranjo, E., Redondo-
Gomez, S., Camacho, M., Caviedes, M. A., Pajuelo, E., and Rodriguez-
Llorente, I. D. (2016). Assessing the role of endophytic bacteria in the
halophyte Arthrocnemum macrostachyum salt tolerance. Plant Biol. 19,
249-256.
Numan, M., Bashir, S., Khan, Y., Mumtaz, R., Shinwari, Z. K., Khan,
A. L., Khan, A., and AL-Harrasi, A. (2018). Plant growth promoting
bacteria as an alternative strategy for salt tolerance in plants: a review.
Microbiol. Res. 209, 21-32.
Oksanen, J. Guillaume, F., Kindt, B., Kindt. R., Legendre, P., Minchin,
P. R., O'Hara, R. B., Simpson, G. L., Solymos, P., Henry, M., Wagner,
S., and Wagner, H. 2013. Vegan: Community Ecology Package.
Orhan, F. (2016). Alleviation of salt stress by halotolerant and halophilic
plant growth-promoting bacteria in wheat (Triticum aestivum). Braz. J.
Microbiol. 47, 621-627.
Palacio-Rodriguez, R., Cora-Arellano, J. L., Lopez-Bucio, J., Sanchez-
Salas, J., Muro-Perez, G., Cataneda-Gaytan, G., and Saenz-Mata, J.
(2017). Halophilic rhizobacteria from Distichlis spicata promote growth
and improve salt tolerance in heterologous plant hosts. Symbiosis 73,
179-189.
Pruesse, E., Quast, C., Knittel, K., Fuchs, B. M., Ludwig, W. G., Peplies,
J., et al. (2007). SILVA: A comprehensive online resource for quality
checked and aligned ribosomal RNA sequence data compatible with
ARB. Nucleic. Acids. Res. 35, 7188-7196. doi: 10.1093/nar/gkm864
Quince, C., Lanzen, A., Davenport, R. J., and Turnbaugh, P. J. (2011).
Removing Noise From Pyrosequenced Amplicons. BMC Bioinformatics
12. doi: 10.1186/1471-2105-12-38
Qurashi, A. W., and Sabri, A. N. (2012). Bacterial exopolysaccharide
and biofilm formation stimulate chickpea growth and soil aggregation
under salt stress. Braz. J. Micro. 2012, 1183-1191.
R Development Core Team, 2018. R: A language and environment for
statistical computing. R Foundation for Statistical Computing, Vienna,
Austria, URL http://www.R-project.org.
Rajput, L., Imran, A., Mubeen, F., and Hafeez, F. Y. (2013). Salt-
tolerant PGPR strain Planococcus rifietoensis promotes the growth and
yield of wheat (Triticum aestivum L.) cultivated in saline soil. Pakistan
J. Bot. 45, 1955-1962.
Ruppel, S., Franken, P., and Witzel, K. (2013). Properties of the
halophyte microbiome and their implications for plant salt tolerance.
Functional Plant Biol. 40, 940-951.
Sanchez-Porro, C., de la Haba, R. R., Soto-Ramirez, N., Marquez, M. C.,
Montalvo-Rodriguez, R., and Ventosa, A. (2009). Description of
Kushneria aurantia gen. nov., sp. nov., a novel member of the family
Halomonadaceae, and a proposal for reclassification of Halomonas
marisflavi as Kushneria marisflavi comb. nov., of Halomonas indalinina
as Kushneria indalinina comb. nov. and of Halomonas avicenniae as
Kushneria avicenniae comb. nov. Intl. J. Sytem. Evol. Micro. 59,
397-405.
Santoyo, G., Moreno-Hagelsieb, G., Orozco-Mosqueda, M. D. C., and
Glick, B. R. (2016). Plant growth-promoting bacterial endophytes.
Microbiol. Res. 183, 92-99.
Schloss, P. D., Westcott, S. L., Ryabin, T., Hall, J. R., Hartmann, M.,
Hollister, E. B., et al. (2009). Introducing mothur: Open-source,
platform-independent, community-supported software for describing and
comparing microbial communities. Appl. Environ. Microbiol. 75, 7537-
7541. doi: 10.1128/AEM.01541-09
Shabala, S. (2013). Learning from halophytes: physiological basis and
strategies to improve abiotic stress tolerance in crops. Annals Bot. 112,
1209-1221.
Shahzad, R., Khan, A. L., Bilal, S., Waqas, M., Kang, S. M., and Lee,
I. J. (2017). Inoculation of abscisic acid-producing endophytic bacteria
enhances salinity stress tolerance in Oryza sativa. Environ. Exp. Bot.
136, 68-77.
Sharma, S., Kulkami, J., and Jha, B. (2016). Halotolerant rhizobacteria
promote growth and enhance salinity tolerance in peanut. Front.
Microbiol. 7, 1600. doi: 10.3389/fmicb.2016.01600
Sukweenadhi, J., Balusamy, S. R., Kim, Y. J., Lee, C. H., Kim, Y. J.,
Koh, S. C., and Yang, D. C. (2018). A growth-promoting bacterium,
Paenibacillus yonginensis DCY84T enhanced salt stress tolerance by
activating defense-related systems in Panax ginseng. Front. Plant Sci. 9,
813. doi: 10.3389/fpls.2018.00813
Turner, S., Pryer, K. M., Miao, V. P. W., and Palmer, J. D. (1999).
Investigating deep phylogenetic relationships among cyanobacteria and
plastids by small subunit rRNA sequence analysis. J. Eukaryot
Microbiol. 46, 327-338.
Vreeland, R. H., Litchfield, C. D., Martin, E. L., and Elliot, E. (1980).
Halomonas elongata, a new genus and species of extremely salt-tolerant
bacteria. Intl. J. System. Bacteriol. 30, 485-495.
Weber, D. J. (2016). The impact of Lake Bonneville and Lake Lanontan
on the halophytes of the Great Basin. In: Sabkha Ecosystems Vol. V:
The Americas. Khan, M. A., Boer, B., Ozturk, M., Clusener-Godt, M.,
Gul, B. (Springer).
Xie, X., Zhang, H., and Pare, P. W. (2009). Sustained growth promotion
in Arabidopsis with long-term exposure to the beneficial soil bacterium
Bacillus subtilis (GB03). Plant Signaling Behavior 4, 948-953.
Yuan, Z., Druzhinina, I. S., Labbe, J., Redman, R., Qin, Y., Rodriguez,
R., Zhang, C., Tuskan, G. A., and Lin, F. (2016). Specialized
microbiome of a halophyte and its role in helping non-host plants to
withstand salinity. Scientific Rep. 6, 32467. doi: 10.1038/srep32467
Zhang, Y. Q., Schumann, P., Yu, L. Y., Liu, H. Y., Zhang, Y. Q., Xu,
L. H., Stackebrandt, E., Jiang, C. L., and Li, W. J. (2007). Zhihengliuella
halotolerans gen. nov., sp. nov., a novel member of the family
Micrococcaceae. Intl. J. System. Evol. Micro. 57, 1018-1023.

TABLE 1

| | Spring (April) 2018 | | Fall (October) 2018 | |
|---|---|---|---|---|
| Plant species | EC dS/m | pH | EC dS/m | pH |
| Allenrolfea and Sarcornia | 16 | 7.56 | 70 | 7.8 |
| Salicornia rubra | 18 | 7.74 | 70 | 7.8 |
| Bare-no plants | 45 | 7.98 | 100 | 7.7 |

TABLE 2

| Genus/species | | | | Max. salt | Temp. | Colony pigment/ | Biofilm | Gram stain/ |
|---|---|---|---|---|---|---|---|---|
| and accession no. | Family | Order | Phylum | tolerance | range ° C. | morphology | formation | cell morphology |
| [symbol] MKB75900 | Micrococcacea | Actinomycetales | Actino- | [symbol] M | N/A | Shiny yellow | ** | Gram + very |
| | | | bacteria | | | | | short rods |
| Halomonas siongata | Halomonadaceae | Oceanospir [symbol] lales | Gamma- | 4M | 22-42 | White, shiny | *** | Gram − short |
| MK [symbol] 73884 | | | Proteo- | | | | | rods |
| | | | bacteria | | | | | |
| Bacillus sp. | Bacillaceae | Baccillales | Firmicutes | [symbol] M | N/A | Dull orange | * | Gram + short |
| MK873882 | | | | | | | | fat rods |
| Virgoacillus sp. | Bacillaceae | Baccillales | Firmicutes | 3M | N/A | White | N/A | Gram + rods |
| MK [symbol] 73894 | | | | | | | | |
| Kushnata marisflavi | Halomonadaceae | Oceanospirallales | Gamma- | 3M | N/A | Red-orange, | *** | Gram − short |
| MK873879 | | | Proteo- | | | shiny | | stubby rods |
| | | | bacteria | | | | | |
| Halomonas huangh [symbol] nsis | Halomonadaceae | Oceanospirallales | Gamma- | 3M | 22-42 | Brown large, | ** | Gram − rods, |
| MK873908 | | | Proteo- | | | shiny | | very short, |
| | | | bacteria | | | | | nearly oval |
| Bacillus licheniformis | Bacillaceae | Baccillales | Firmicutes | 3M | 22-42 | White round, | ** | Gram + long rods |
| MK873893 | | | | | | flat | | |
| Bacillus so [symbol] | Bacillaceae | Baccillales | Firmicutes | 1.5M | 22-42 | Dull yellow | ** | Gram + long |
| MK879902 | | | | | | small, round | | filamentous rods |

Genbank accession numbers are provided below the most probable genus and species name of each isolate.
Biofilm formation is characterized as:
—, no detectable biofilm,
* detectable but low level of biofilm,
** moderate biofilm formation,
*** strong biofilm formation. [symbol]
indicates data missing or illegible when filed

What is claimed is:

1. An artificial salt tolerant plant comprising;
a glycophyte plant combined with a non-host halophile bacteria inoculated into the glycophyte plant rhizosphere or as an endophyte to form the salt tolerant plant, the salt tolerant plant having a symbiotic relationship between the plant and the non-host halophile bacteria to provide growth promotion to the formed salt tolerant plant under saline conditions and to form an artificial plant/bacteria combination that does not naturally occur,
the non-host halophile bacteria being identifiable as a naturally occurring soil bacteria associated with a halophyte plant, the halophyte plant being identifiable as a member of inland occurring halophyte plants of the subfamily Salicornioideae.

2. The plant of claim 1 wherein the halophyte plant is of genus Allenrolfea, or genus Salicornia, or genus Sarcocornia.

3. The plant of claim 1, wherein the halophyte plant is Allenrolfea occidentalis, Salicornia rubra, or Sarcocornia utahensis.

4. The plant of claim 1, wherein the bacteria is from genus Halomonas, Kushneria, or Bacillus.

5. The plant of claim 1, wherein the bacteria have at least one sequence recorded in GenBank under accession numbers MK873873 to MK873913.

6. The plant of claim 1, wherein the bacteria have one or more of the Illumina sequence reads available at the NCBI Sequence Archive under BioProject ID PRJNA553550, BioSample accessions SAMN12238110, SAMN12238111, SAMN12238112, SAMN12238113, SAMN12238114, SAMN12238115, SAMN12238116, SAMN12238117, SAMN12238118, SAMN12238119.

7. The plant of claim 1, wherein the glycophyte plant is from alfalfa, Kentucky blue grass, or Bermuda grass.

8. The plant of claim 1, wherein the glycophyte plant is from a grass.

9. The plant of claim 8, wherein the grass is from a turf grass.

10. The plant of claim 9, wherein the turf grass is from Kentucky blue grass, or Bermuda grass.

11. A method for creating an artificial salt tolerant plant comprising:
inoculating a glycophyte plant with a non-host halophile bacteria into the plant rhizosphere or as an endophyte to form the salt tolerant plant, the salt tolerant plan having a symbiotic relationship between the plant and the non-host halophile bacteria to provide growth promotion to the salt tolerant plant under saline conditions, the symbiotic relationship being an artificial plant/bacteria combination that does not otherwise naturally occur, the non-host halophile bacteria being identifiable as a naturally occurring soil bacteria associated with a halophyte plant, the halophyte plant being identifiable as a member of inland occurring halophyte plants of the subfamily Salicornioideae.

12. The method of claim 11, wherein the halophyte plant is of genus Allenrolfea, or genus Salicornia, or genus Sarcocornia

13. The method of claim 12, wherein the halophyte plant is Allenrolfea occidentalis, Salicornia rubra, or Sarcocornia utahensis.

14. The method of claim 11, wherein the bacteria is from genus Halomonas Kushneria, or Bacillus.

15. The method of claim 11, wherein the bacteria have at least one sequence recorded in GenBank under accession numbers MK873873 to MK873913.

16. The method of claim 11, wherein the bacteria have one or more of the Illumina sequence reads available at the NCBI Sequence Archive under BioProject ID PRJNA553550, BioSample accessions SAMN12238110, SAMN12238111, SAMN12238112, SAMN12238113, SAMN12238114, SAMN12238115, SAMN12238116, SAMN12238117, SAMN12238118, SAMN12238119.

17. The method of claim 11, wherein the glycophyte plant is from alfalfa, Kentucky blue grass, or Bermuda grass.

18. The method of claim 11, wherein the glycophyte plant is from a grass.

19. The method of claim 18, wherein the grass is from a turf grass.

20. The method of claim 19, wherein the turf grass is from Kentucky blue grass, or Bermuda grass.
